(19) United States
(12) Patent Application Publication
(10) Pub. No.: US 2013/0218113 A1
(43) Pub. Date: Aug. 22, 2013

(54) ABSORBENT PAD FOR ADULT ACTIVITIES

(71) Applicant: Erin Strathdee, Atlanta, GA (US)

(72) Inventor: Erin Strathdee, Atlanta, GA (US)

(21) Appl. No.: 13/398,571

(22) Filed: Feb. 16, 2012

(51) Int. Cl. A61L 15/46

(52) U.S. Cl. 604/359

(57) ABSTRACT

Embodiments disclosed herein provide an absorbent pad for use during adult activity having multiple layers of flexible materials including, an outer layer disposed along a first side of the pad formed of a material permeable to liquids; an intermediate layer comprising a scented material and semi-permeable to liquids; a further intermediate layer comprising an absorbent material capable of absorbing and trapping liquids; and an outer layer disposed along a second side of the pad that is impermeable to liquids. Disposed along the surface of the outer layer is an adhesive layer for locking the pad in place.

### BACKGROUND OF THE INVENTION

1. Field of the Technology

The present invention relates to an absorbent article, such as an absorbent pad for personal use, more particularly, it relates to an absorbent pad designed for adult activity to absorb fluids and to prevent damage to linens, furniture and other items while also providing aroma therapy.

2. Background

Absorbent pads of the prior art have a broad range of applications and are configured and constructed for being worn for extend periods of time to contain the exudates of a child or an adult. Absorbent pads typically include a liquid permeable layer adjacent to the skin of the wearer, one or more absorbing layers, and an exterior liquid impermeable backing. Absorbent pads often have fasteners, such as adhesive portions, to the pads, such as diapers, in place while they are worn. Many absorbent pads also include one or more biasing members to prevent the pads from shifting around while being worn in a person's underwear, thereby retaining the pad in the optimal position for capturing liquids.

Absorbent pads of the prior art also include pads for capturing animal or pet waste, for placement in hospital beds for incapacitated patients. It is customary to use an absorbent pad between the patient and mattress of the bed in order to protect the mattress. In the case of simple impervious material such as rubber or plastic, which is usually placed between the mattress and bed linens, the mattress is protected, but not the bed linens. Once the patient has urinated, the patient may be subjected to discomfort since the bed linens become wet and retain the smell of urine.

While absorbent hospital bed pads are known the art for protecting patients from wetness, there exists no product for consumer use. Busy adult couples often have to take advantage of opportunities to connect for adult sexual activity. However, many adults do not wish to change bed linens or clean up other areas of their home, vehicle, or personal spaces after sexual activity. Still, these adults do not desire to allow a moment of passion to pass for convenience sake. Therefore, it would be desirable to have a liquid absorbent, portable, sanitary product that can be draped over beds, sofas, chairs, car seats, floors and other surfaces during sexual activity to prevent wetness, stains, and odors.

### SUMMARY

Embodiments disclosed herein provide an absorbent pad for use during sexual activity having multiple layers of flexible materials including, an outer layer disposed along a first side of the pad formed of a material permeable to liquids; an intermediate layer comprising a scented material and permeable to liquids; a further intermediate layer comprising an absorbent material capable of absorbing and trapping liquids; and an outer layer disposed along a second side of the pad that is impermeable to liquids. Disposed along the surface of the outer layer is an adhesive layer for locking the pad in place.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims or may be learned by the practice of the invention as set forth hereinafter.

### BRIEF DESCRIPTION OF THE DRAWINGS

FIG. 1 is a cross-sectional view of an absorbent pad, according to an embodiment of the invention;

FIG. 2 is a cross-sectional view of an aroma therapy pad insert, according to an embodiment of the invention; and

FIG. 3 is a view of an adhesive locking surface of the absorbent pad according to an embodiment of the invention, according to an embodiment of the invention;

### DETAILED DESCRIPTION OF EXEMPLARY EMBODIMENTS

For purposes of promoting an understanding of the principle of the invention, reference will now be made to the exemplary embodiments illustrated in the drawing(s), and specific language will be used to describe the same. It will never the less be understood that no limitation of the scope of the invention is thereby intended. Any alterations and further modification of the inventive features illustrated herein, and any additional application of the principles of the invention as illustrated herein, which would occur to one skilled in the relevant art and having a possession of this disclosure, are to be considered within the scope of the invention.

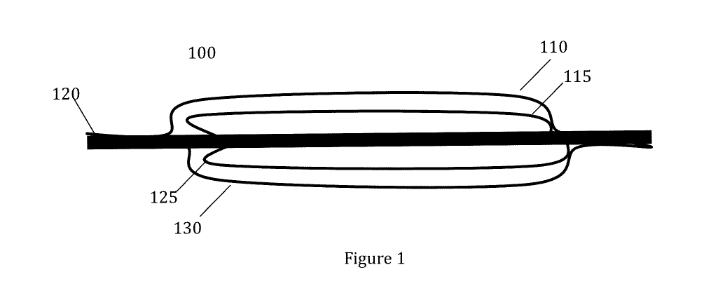

A cross section of view of an absorbent pad is indicated in FIG. 1. The absorbent pad 100 is designed to be placed between the user(s) and a solid surface, such as a bed, other piece of furniture, car seat, floor, etc. The absorbent pad 100 may come in a variety of shapes and sizes, however, in an exemplary embodiment; the absorbent pad is comparable size of a full size bed sheet. As shown in the cross-section illustration of FIG. 1, the absorbent pad 100 is comprised of a permeable layer 110, an aromatherapy layer 115, an absorbent layer 120, an impermeable layer 125 and finally an adhesive layer 130. The permeable layer covers an entire top surface of the pad which is designed to allow fluids to seep through to the absorbent layer 120 and away from the user(s).

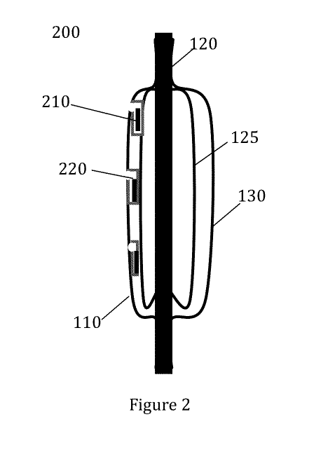

As illustrated in FIG. 1, between the permeable layer 110 and the absorbent layer 120 is an aroma therapy layer 115. The aroma therapy layer 115 is a semi-permeable pad that is designed to allow liquids too seep through in one direction away from the permeable layer 110 to the absorbent layer 120, such that, when liquids pass through the aroma therapy layer 115, they do not seep back up to the user(s). The aroma therapy layer 115 is also a scented or fragrant component that release fragrant aromas in response to friction, heat, liquids and/or other motions. The permeable nature of the permeable layer 110 allows the aromas from the aroma therapy layer to seep out so the user(s) can enjoy relaxing aromas while using the absorbent pad 100. In another embodiment, illustrated in FIG. 2, the absorbent layer 115 may be comprised of removable aroma therapy pads 210 that may be placed in pockets 220 fused into the permeable layer 110. Such an embodiment may allow the absorbent pad 100 to be shipped with a variety of aromatic pads 210 that could be selected to a user's taste and inserted into the pockets 220 disposed along the permeable layer 110.

Further illustrated in FIG. 1, along the center of the pad is an absorbent layer 120. The absorbent layer 120 may be comprised of any absorbent natural or synthetic fiber, wool fluff, woven or nonwoven coarse material, cellulosic bats, and/or open-cell sponge-like sheets. This highly absorber layer 120 is designed to absorb and hold fluids and liquids that's may result from adult sexual activity. Therefore, the absorbent layer 120 may comprises a plurality of layers of absorbent materials. In an embodiment, the materials in the absorbent layer 120 are loosely packed such that the absorbent layer 120 may receive fluids from a variety of viscosities.

Next below the absorbent layer 120 is an impermeable layer 125 for backing the absorbent layer 120. The impermeable layer 125 may be comprised of moisture barrier film that is impervious to fluids and liquids, such as a polyethylene or polypropylene film, or the like. The impermeable layer 125 is designed to be durable enough to handle the rigors of stretching and pulling during adult physical activity while maintaining the overall structure of the absorbent pad 100. The impermeable layer 125 may also contain logos, advertisements, patterns, colors and other indicia which may be customizable to a user or product supplier.

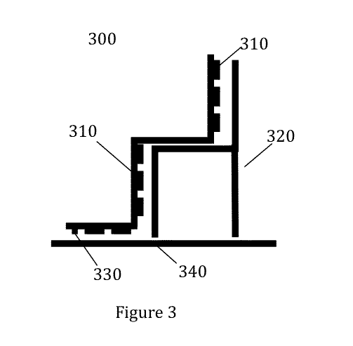

Disposed on the surface of the impermeable layer 125 is an adhesive layer 130. The adhesive layer may comprise a light, non-permanent adhesive that allows the pad to stick to a surface without moving around. Similarly, as illustrated in FIG. 3, Velcro® or a similar hook and loop system 310 may also be disposed on the surface of the impermeable layer 125. In an embodiment, several strips of a hook system 310 may be disposed along the impermeable layer 125. The hook strips may be designed to frictionally engage a fabric surface 320 such as a sofa, bed sheets or a car seat. Similarly, a light adhesive 330 may be designed to engage a fabric, wood, plastic or other surface 340 while being easily removable without leaving any adhesive residue on the surface. There may also be a combination of hooks 310 and adhesive strips 330 disposed on the surface of the impermeable layer 125 as illustrated in FIG. 3.

Each of the above described layers may be fused together along an outer perimeter of the absorbent pad 100 using a suitable adhesive and/or heat sealing means. Thus the pad may be appear as a large, flat pillow, being sealed along the perimeter and filled throughout the center with the various layers.

Thus, while the present invention has been fully described above with particularity and detail in connection with what is presently deemed to be the most practical and preferred embodiment of the invention, it will be apparent to those of ordinary skill in the art that numerous modifications, including but not limited to, variations in size, materials, shape, form, and function and manner of operation, assembly and use may be made without departing from the principles and concepts of the invention as set forth in the claims. Further, it is contemplated that an embodiment may be limited to consist of, or to consist essentially of one or more of the features, functions, structures, methods, described herein.

What is claimed is:

1. An absorbent pad for use during adult activity having multiple layers of flexible materials including:
a permeable outer layer disposed along a first side of the pad formed of a material permeable to liquids;
an intermediate layer comprising a scented material and permeable to liquids;
a further intermediate layer comprising an absorbent material capable of absorbing and trapping liquids;
an impermeable backing layer disposed along a second side of the pad that is impermeable to liquids; and
an adhesive layer distributed along a surface of the outer layer disposed along the second side of the pad for locking it in place.

2. The absorbent pad of claim 1, wherein the permeable outer layer of the pad is designed to be used against a user's skin.

3. The absorbent pad of claim 1, wherein said scented material is responsive to heat, friction and/or movement.

4. The absorbent pad of claim 1, wherein said absorbent material comprises cotton, wool, fabric or other cellulose components.

5. The absorbent pad of claim 1, wherein said absorbent material comprises a gel emulsification component.

6. The absorbent pad of claim 1, wherein said adhesive layer comprises a non-binding adhesive that is easily removed.

7. The absorbent pad of claim 1, wherein said adhesive layer comprises a fabric hook system for locking the pad in place against a fabric surface.

8. The absorbent pad of claim 1, wherein said intermediate layer comprises one of more pockets disposed below the permeable outer layer for receiving aroma therapy pads.

9. A system for providing an absorbent pad for use during adult activity having multiple layers of flexible materials including:
a means for receiving fluid along a first surface of an absorbent pad;
a means for releasing scented fragrances in response to activity on the absorbent pad;
a means for capturing and storing fluids along one layer of an absorbent pad;
a means for providing an impermeable backing layer disposed along a second surface of the pad that is impermeable to liquids; and
a means for providing an adhesive layer distributed along the second surface of the pad for locking it in place.

10. The system of claim 9, wherein the first surface of the pad is designed to be used against a user's skin.

11. The system of claim 9, wherein the means for releasing scented fragrances is responsive to heat, friction and/or movement.

12. The system of claim 9, wherein the means for capturing and storing fluids comprises absorbent material including at least one of cotton, wool, fabric or other cellulose components.

13. The system of claim 9, wherein the means for capturing and storing fluids comprises a gel emulsification component.

14. The system of claim 9, wherein the means for providing an adhesive layer comprises a non-binding adhesive that is easily removed.

15. The system of claim 9, wherein the means for providing an adhesive layer comprises a fabric hook system for locking the pad in place against a fabric surface.

16. The system of claim 9, wherein the means for releasing scented fragrances comprises one of more pockets disposed below the permeable outer layer for receiving aroma therapy pads.

17. A system for providing an absorbent pad for use during adult activity having multiple layers of flexible materials including:
a means for receiving fluid along a preamble first surface of an absorbent pad, wherein the first surface includes include a means for storing scented pads that release fragrances in response to activity on the absorbent pad;
a means for capturing and storing fluids along one layer of an absorbent pad;
a means for providing an impermeable backing layer disposed along a second surface of the pad that is impermeable to liquids; and
a means for providing an adhesive layer distributed along the second surface of the pad for locking it in place.

18. The system according to claim 17, wherein the means for storing scented pads includes sealable pockets disposed along the preamble first surface of the absorbent pad.

19. The system according to claim 17, wherein the scented pads adhesively attach to the permeable first surface of the absorbent pad.

20. The system according to claim 17, wherein the scented pads are permeable to fluids.
